(19) United States
(12) Patent Application Publication
(10) Pub. No.: US 2020/0171255 A1
(43) Pub. Date: Jun. 4, 2020

(54) EFFECTIVELY-SUPPORT-TYPE OXYGEN INHALATION AND SPUTUM ASPIRATION OROPHARYNGEAL AIRWAY DEVICE

(71) Applicant: Jinfang Xiao, Guangzhou (CN)

(72) Inventors: Jinfang XIAO, Guangzhou (CN); Xinsheng HUANG, Guangzhou (CN); Wenqi HUANG, Guangzhou (CN); Yinming ZENG, Guangzhou (CN); Miaoning GU, Guangzhou (CN)

(21) Appl. No.: 16/638,126

(22) PCT Filed: Aug. 22, 2017
(86) PCT No.: PCT/CN2017/098521

§ 371 Date: Feb. 10, 2020

(30) Foreign Application Priority Data
Aug. 10, 2017 (CN) 201710683535.7

(51) Int. Cl. A61M 16/04

(52) U.S. Cl. CPC A61M 16/0463; A61M 16/0497

(57) ABSTRACT

An effectively-support-type oxygen inhalation and sputum aspiration oropharyngeal airway device, comprising an inner airway body provided from a proximal end to a distal end, and a support plate parallel tongue surface and having -a-certain width; the support plate comprises a proximal end positioned on the front face of inner airway body and constituting the wall of duct, and a distal bending portion with bending angle of 0°-180°; the front face of distal bending portion is provided with at least two evenly distributed projections, recesses, or through holes; the surfaces of the projections facing to teeth are smooth and the surfaces of facing to pharynx have a height of 1-2 mm, or the recessing depth of recesses is 0.5-1 mm, or the diameter of through holes is 1-3 mm; the inner airway body provided from a proximal end to a distal end integrates a sputum aspiration duct and an oxygen inhalation duct.

### CROSS-REFERENCE TO RELATED APPLICATIONS

The present disclosure claims the priority of the Chinese Patent Application No. 201710683535.7, entitled “Effectively-support-type Oxygen Inhalation and Sputum Aspiration Oropharyngeal Airway Device”, filed with the Chinese Patent Office on Aug. 10, 2017, the entity of which is incorporated herein by reference.

### TECHNICAL FIELD

The present disclosure relates to the technical field of medical instruments, and particularly to an effectively-support-type oxygen inhalation and sputum aspiration oropharyngeal airway device (i.e., efficient care-type oropharyngeal airway device permitting oxygen inhalation and sputum aspiration).

### BACKGROUND

Oropharyngeal airway devices are commonly used during the recovery period from surgical general anesthesia. During the anesthesia recovery period, due to the residual of anesthetic drugs and muscle relaxants or the subsequent effects of metabolites thereof, patients are in blurred state of consciousness or are drowsy, even some undergo delayed recovery, and this state renders relaxation of oropharyngeal muscle groups, especially the tongue, as tongue muscle relaxation would easily cause glossocoma, resulting in narrowness or stenosis of the pharyngeal cavity or total occlusion of the pharyngeal cavity, which may cause obstructed airway of the patients, and thereby easily cause disturbance of ventilation or total loss of ventilation function of the patients during the recovery period from surgical general anesthesia, wherein oxygen supply and oxygen consumption of human body are out of balance, and the most severe case is the death of patients because of postoperative hypoxia. The case mentioned above is shown in FIG. 25.

Traditional oropharyngeal airway devices can solve the problems of relaxation of oral muscle groups caused by blurred state of consciousness or drowsiness or delayed recovery of patients during the recovery period from surgical general anesthesia, and can effectively support tongues fell afterwards, and establish a new respiration channel during the recovery period from surgical general anesthesia.

However, the recovery of patients undergoing the recovery period from surgical general anesthesia requires time, and along with the gradual degradation of anesthetic drugs and metabolism of the patient's body from the liver, kidney and lung as well as the metabolism of various biological enzymes, the molecular structure of anesthetic is destroyed or eliminated; and the sense and consciousness of the patient gradually become clearer and recovered as the anesthetic is gradually metabolized and eliminated. Moreover, along with the recovery of this consciousness and sense, the patient cannot tolerate the stimulation caused by the traditional oropharyngeal airway device, especially, adverse reflexes, such as nausea and retching, might be caused, when some sensitive regions in the pharyngeal cavity are stimulated by the traditional oropharyngeal airway device, and nausea and vomiting might even occur in severe cases. In this case, a great amount of secretions in the stomach produced during the surgery might flow backwards or be sprayed into the oral cavity with the increase of abdominal pressure, and then might be accidentally inhaled into the tracheal bronchi and the lung of the airway. As the gastric contents are acidic secretion with a pH value of 0.9-3, these acidic contents immediately lead to the inactivation of alveolar surfactant, dipalmityl lecithin, and damage of alveolar cells; tracheobronchial spasm and blockage of reflux contents, wherein not only disturbance of ventilation arises in the human body, but the patient would also suffer from aeration disorder, secondary atelectasis, pulmonary edema, and lung infections.

In addition, abdominal muscles might contract acutely because of nausea and retching caused by stimulation of the traditional oropharyngeal airway device, which might result in wound splitting. Stimulation caused by the use of the traditional oropharyngeal airway device might cause hypersensitive reaction of the airway, and result in obvious secondary cyclic dynamic fluctuations, which indicates increased blood pressure and increased heart rate, easily resulting in would bleeding during surgery of the liver and the brain, and thereby causing failure of surgery. And patients suffering from diabetes, hypertension, adiposis, snoring disease and the like may easily undergo related complications, leading to heart diseases, cerebrovascular accidents, and recurrence of disturbance of ventilation such as airway obstruction.

### SUMMARY

An object of the present disclosure is to provide an efficient care-type oropharyngeal airway device permitting oxygen inhalation and sputum aspiration, so as to solve the problem that an existing oropharyngeal airway device may have an adverse stimulation effect, which would easily cause the occurrence of complications endangering the life of a patient.

An embodiment of the present disclosure is implemented through the following technical solution:

An efficient care-type oropharyngeal airway device permitting oxygen inhalation and sputum aspiration, comprising: an internal airway body extending from a proximal end to a distal end, and a support plate having a width and parallel to the lingual surface of the human body, wherein the support plate includes a proximal end and a distal end bending section, with the proximal end being located at the front side of the internal airway body and constituting a wall of the tube, and the distal end bending section having a bending angle of 0°-180°, wherein the front side of the distal end bending section is provided with at least two uniformly distributed projections or recesses or through holes, with the surface of the projection facing to the teeth being smooth and the surface thereof facing to the pharynx having a height of 1-2 mm, or the depth of the recess being 0.5-1 mm or the diameter of the through hole being 1-3 mm.

The purpose of the above design is to change the adhesion factor or the coefficient of friction surface between the support plate of the efficient care-type oropharyngeal airway device permitting oxygen inhalation and sputum aspiration and the lingual surface, so as to prevent the tongue from falling off the plate and then falling afterward.

Further, the width of the proximal end of the support plate is greater than the width of the distal end bending section; and an edgefold is respectively formed at both sides of the proximal end, and the included angle is in the range of 0°-180°.

The purpose of such a design is to form an enveloping state on the body of the tongue.

Further, the efficient care-type oropharyngeal airway device permitting oxygen inhalation and sputum aspiration further comprises an oxygen inhalation tube, which is arranged on a side wall of the internal airway body.

Further, a side wall of the oxygen inhalation tube is provided with at least two side holes that are spaced from each other, the plurality of side holes are distributed along the proximal end of the internal airway body to the distal end, and a bottom hole at the distal end of the oxygen inhalation tube is closed; and the perforated angle of the side hole has an included angle of 0°-180° with respect to any axial lead.

The purpose of such a design is to enable the formation of a physiologic state of mixed flow of the inhaled gas after its entry into the trachea, so as to prevent that direct air blowing towards a certain part of the airway for a long time causes the dehydration of local tissue.

Further, the efficient care-type oropharyngeal airway device permitting oxygen inhalation and sputum aspiration further comprises a sputum aspiration tube, which is arranged on the side wall of the reverse side of the internal airway body and has a distal opening serving as sputum aspiration hole.

The sputum aspiration hole is provided at the bottom of the tube, which is conducive to the accumulation of sputum in the opening of the sputum aspiration hole, around the bottom of the tube and thus facilitates the sputum aspiration operation, since the pharyngeal cavity is in low position, when the patient is supine.

Further, the efficient care-type oropharyngeal airway device permitting oxygen inhalation and sputum aspiration further comprises an independent air pocket, which is positioned on the reverse side of the internal airway body and is in connection with an inflation valve.

The purpose of such a design lies in that the sustentive support plate is able to lift the lingual surface and raise the root of tongue, when the independent air pocket is inflated, such that the space of the pharyngeal cavity is enlarged, which is conductive to an unobstructed airway of patents during the anesthesia recovery period; meanwhile, the inflated independent air pocket abuts against the hard palate part, which is less sensitive and is hard to irritate.

Further, the efficient care-type oropharyngeal airway device permitting oxygen inhalation and sputum aspiration further comprises a double-layered air pocket, which is positioned on the reverse side of the internal airway body and constitutes the rear wall of the internal airway body.

The effect and function of such a design are similar to that of the independent air pocket when being inflated; and in order to reduce material consume, the double-layered air pocket is enabled to constitute the rear wall of the internal airway body.

Further, the internal airway body has a circular cross-section or an elliptical cross-section or a special-shaped cross-section.

An efficient care-type oropharyngeal airway device permitting oxygen inhalation and sputum aspiration, comprising: an internal airway body extending from a proximal end to a distal end, and a support plate having a width and parallel to the lingual surface of the human body, wherein the support plate has a plate-like proximal end, and the both sides of the plate are infolded with an infolding radian of 0°-180°, while the distal end of the support plate is a curved arc structure with a bending radian of 0°-180°; the curved arc structure of the distal end of the support plate is provided with an tongue adhering protuberance, which includes a through hole, projection or recess; the internal airway body has a length having a ratio of 1.5/3-2.5/3 with respect to the overall length of the efficient care-type oropharyngeal airway device permitting oxygen inhalation and sputum aspiration; the internal airway body is in a hard circular or elliptical structure, or the internal airway body is in a soft special-shaped structure; the internal airway body is in communication with an inflation valve, and the reverse side of the hard circular or elliptical structure is provided with an inflation pocket constituting a tubal cavity part and being in communication with the inflation valve; alternatively, the soft special-shaped structure is adhered to the hard part and is in communication with a tubal ring and the inflation valve, and constitutes a tube wall of the efficient care-type oropharyngeal airway device permitting oxygen inhalation and sputum aspiration when being inflated; a side tube wall of the internal airway body is provided with an oxygen inhalation tube, which is provided with at least two side holes, wherein every two airflows respectively between two adjacent side holes form an included angle therebetween and converge in the internal of the internal airway body to form a turbulence; the bottom of the oxygen inhalation tube is closed; and the back tube wall of the internal airway body is provided with a sputum aspiration tube.

Compared with the prior art, the technical solutions of the present disclosure have at least the following advantages or beneficial effects:

The efficient care-type oropharyngeal airway device permitting oxygen inhalation and sputum aspiration provided in the embodiments of the present disclosure can effectively avoid regions in the oropharyngeal cavity sensitive to stimulus, that is to say, it can effectively avoid that regions having a reflected intensity higher than 5 are stimulated, hereby minimizing stimulation area of the sensitive regions mentioned above and reducing stimulation intensity during the anesthesia recovery period. Compared with the traditional oropharyngeal airway device, the efficient care-type oropharyngeal airway device permitting oxygen inhalation and sputum aspiration provided in the embodiments of the present disclosure is safe and efficient in use, and its stimulation area is in a non-sensitive reflection region, hereby reducing the occurrence of nausea and vomiting as well as choking cough, while ensuring an unobstructed glottal upper airway during the anesthesia recovery period. It is especially suitable for patients underwent surgery with general anesthesia, who have acromegaly, snoring disease, glossocoma after extubation and the like, and its purpose is to reduce adverse reactions during the postanesthetic recovery period, conforming to the tool sequential extubation method and the idea of comfort care.

### BRIEF DESCRIPTION OF THE DRAWINGS

In order to more clearly describe the technical solutions in the specific embodiments of the present disclosure or in the prior art, the figures to be used in the description of the specific embodiments or the prior art will be simply presented below; and obviously, the figures in the following description show certain special embodiments of the present disclosure, and for a person ordinarily skilled in the art, further figures could be obtained according to these figures without inventive efforts, and other figures not illustrated shall all be covered in the scope of protection of the present disclosure.

FIG. 1 shows a structural schematic diagram of the front part of an efficient care-type oropharyngeal airway device permitting oxygen inhalation and sputum aspiration provided in an embodiment of the present disclosure;

FIG. 2 shows a structural schematic diagram of the rear part of the efficient care-type oropharyngeal airway device permitting oxygen inhalation and sputum aspiration provided in an embodiment of the present disclosure;

FIG. 3 shows a structural schematic diagram of the front part of another efficient care-type oropharyngeal airway device permitting oxygen inhalation and sputum aspiration provided in an embodiment of the present disclosure;

FIG. 4 shows a structural schematic diagram of the rear part of the another efficient care-type oropharyngeal airway device permitting oxygen inhalation and sputum aspiration provided in an embodiment of the present disclosure;

FIG. 5 shows a frontal structural schematic diagram of the support plate in an efficient care-type oropharyngeal airway device permitting oxygen inhalation and sputum aspiration provided in an embodiment of the present disclosure;

FIG. 6 shows a structural schematic diagram of through holes of the support plate in an efficient care-type oropharyngeal airway device permitting oxygen inhalation and sputum aspiration provided in an embodiment of the present disclosure;

FIG. 7 shows a structural schematic diagram of projections of the support plate in an efficient care-type oropharyngeal airway device permitting oxygen inhalation and sputum aspiration provided in an embodiment of the present disclosure;

FIG. 8 shows a structural schematic diagram of recesses of the support plate in an efficient care-type oropharyngeal airway device permitting oxygen inhalation and sputum aspiration provided in an embodiment of the present disclosure;

FIG. 9 shows a structural schematic diagram of the cross-section of the proximal end of another efficient care-type oropharyngeal airway device permitting oxygen inhalation and sputum aspiration provided in an embodiment of the present disclosure;

FIG. 10 shows a structural schematic diagram of the oxygen inhalation tube in an efficient care-type oropharyngeal airway device permitting oxygen inhalation and sputum aspiration provided in an embodiment of the present disclosure;

FIG. 11 shows a structural schematic diagram for the implantation of an efficient care-type oropharyngeal airway device permitting oxygen inhalation and sputum aspiration provided in an embodiment of the present disclosure into a mouth cavity;

FIG. 12 shows a structural schematic diagram for an efficient care-type oropharyngeal airway device permitting oxygen inhalation and sputum aspiration provided in an embodiment of the present disclosure approaching the pharyngeal cavity;

FIG. 13 shows a structural schematic diagram of the front part of yet another efficient care-type oropharyngeal airway device permitting oxygen inhalation and sputum aspiration provided in an embodiment of the present disclosure;

FIG. 14 shows a structural schematic diagram of the rear part of the yet another efficient care-type oropharyngeal airway device permitting oxygen inhalation and sputum aspiration provided in an embodiment of the present disclosure in a deflated state;

FIG. 15 shows a structural schematic diagram of the rear part of the yet another efficient care-type oropharyngeal airway device permitting oxygen inhalation and sputum aspiration provided in an embodiment of the present disclosure in an inflated state;

FIG. 16 shows a structural schematic diagram of the cross-section of the proximal end of the yet another efficient care-type oropharyngeal airway device permitting oxygen inhalation and sputum aspiration provided in an embodiment of the present disclosure;

FIG. 17 shows a structural schematic diagram for the implantation of an efficient care-type oropharyngeal airway device permitting oxygen inhalation and sputum aspiration provided in an embodiment of the present disclosure in a deflated state into a mouth cavity;

FIG. 18 shows a structural schematic diagram for the implantation of an efficient care-type oropharyngeal airway device permitting oxygen inhalation and sputum aspiration provided in an embodiment of the present disclosure in an inflated state into a mouth cavity;

FIG. 19 shows a structural schematic diagram of the front part of still another efficient care-type oropharyngeal airway device permitting oxygen inhalation and sputum aspiration provided in an embodiment of the present disclosure;

FIG. 20 shows a structural schematic diagram of the rear part of the still another efficient care-type oropharyngeal airway device permitting oxygen inhalation and sputum aspiration provided in an embodiment of the present disclosure;

FIG. 21 shows a structural schematic diagram of the rear part of the still another efficient care-type oropharyngeal airway device permitting oxygen inhalation and sputum aspiration provided in an embodiment of the present disclosure in an inflated state;

FIG. 22 shows a structural schematic diagram of the cross-section of the proximal end of the still another efficient care-type oropharyngeal airway device permitting oxygen inhalation and sputum aspiration provided in an embodiment of the present disclosure;

FIG. 23 shows a structural schematic diagram for the implantation of the still another efficient care-type oropharyngeal airway device permitting oxygen inhalation and sputum aspiration provided in an embodiment of the present disclosure into a mouth cavity;

FIG. 24 shows a structural schematic diagram for the implantation of the still another efficient care-type oropharyngeal airway device permitting oxygen inhalation and sputum aspiration provided in an embodiment of the present disclosure in an inflated state into a mouth cavity;

FIG. 25 shows a schematic diagram for a muscular flaccidity part in the pharyngeal cavity and a disturbance of ventilation in the airway under anesthesia or during the recovery period; and

FIG. 26 shows a distribution diagram of regions sensitive to stimulus in the oropharyngeal cavity.

In the figures: 11-internal airway body; 10-support plate; 101-proximal end; 102-distal end bending section; 4-tongue adhering protuberance; 401-through hole; 402-projection; 403-recess; 1-oxygen inhalation tube; 2-side hole; 7-sputum aspiration tube; 5-independent air pocket; 6-inflation valve; 9-double-layered air pocket; and 8-tubal ring.

### DETAILED DESCRIPTION OF THE EMBODIMENTS

The technical solutions of the embodiments of the present disclosure will be clearly and comprehensively described below with reference to the figures. Obviously, the described embodiments are merely some of the embodiments of the present disclosure, but not all the embodiments. Any other embodiments, obtained by a person ordinarily skilled in the art based on the embodiments in the present disclosure without inventive efforts, shall fall within the scope of protection of the present disclosure.

In the description of the present disclosure, it shall be clarified that, unless otherwise expressly specified and defined, terms such as “mount”, “link”, and “connect” shall be construed in a broad sense. For example, it may be fixed connection, or detachable connection, or integrated connection; it may be mechanical connection; or it may be direct connection, or indirect connection via an intermediate, or inner communication between two elements. For a person ordinarily skilled in the art, the specific meanings of the above-mentioned terms in the present disclosure could be construed in accordance with specific circumstances.

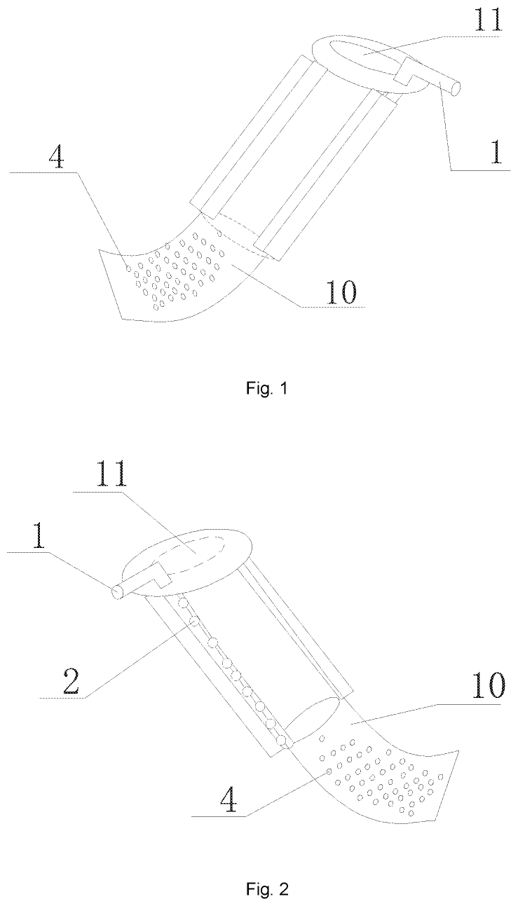

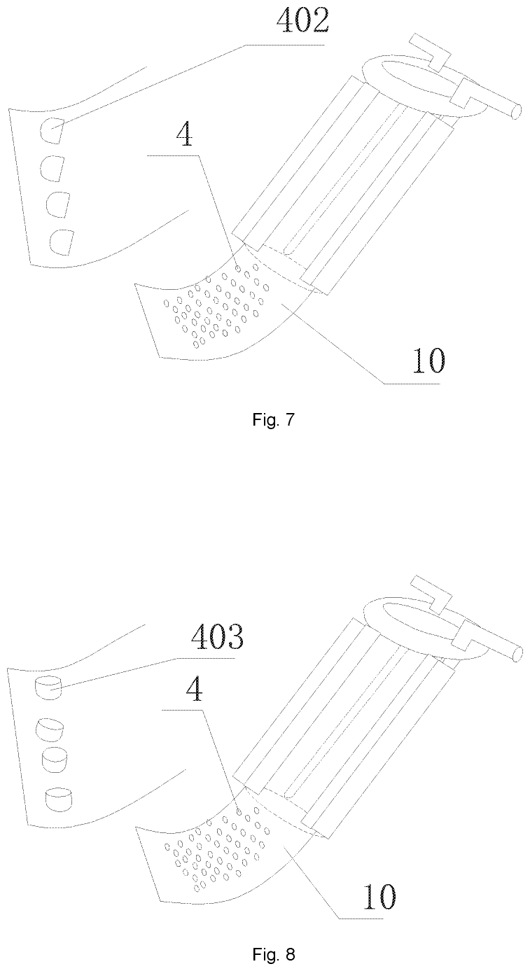

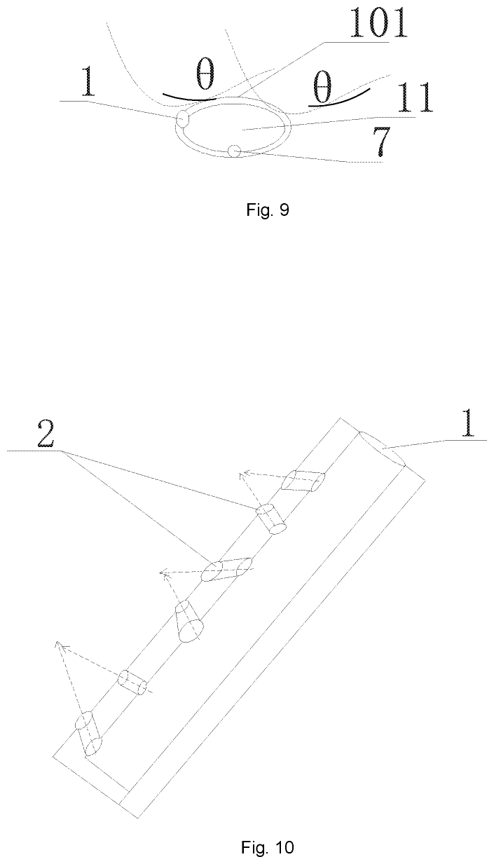

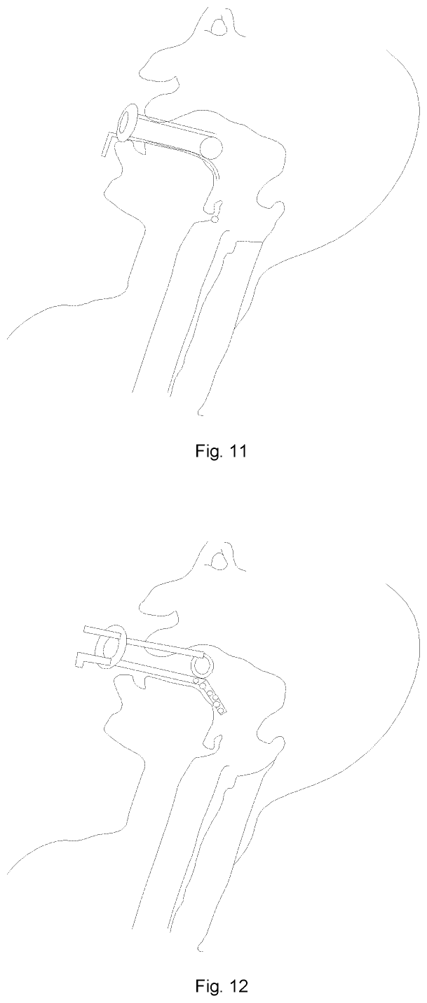

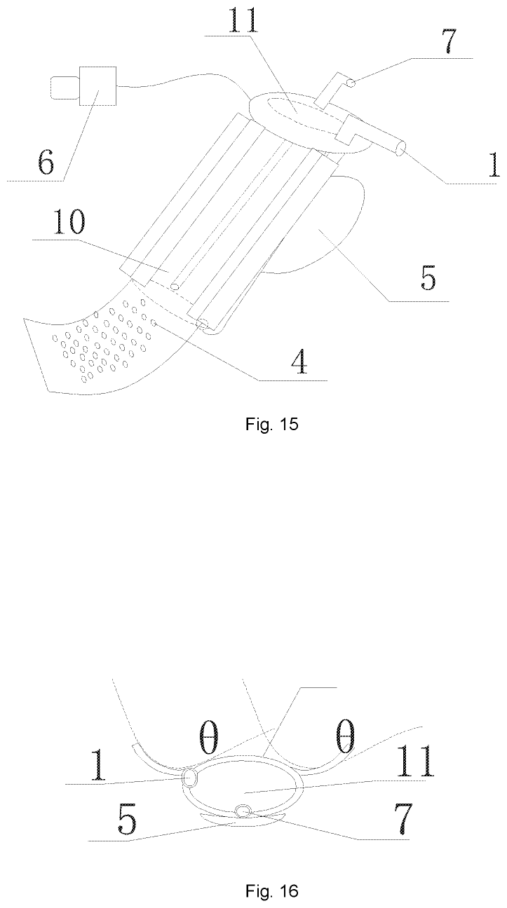

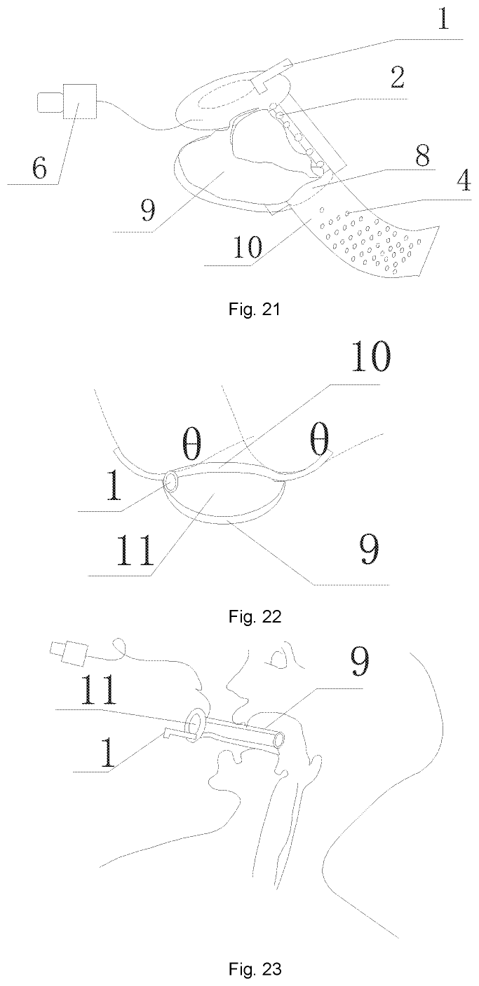

As shown in FIGS. 1-24, an embodiment of the present disclosure provides an efficient care-type oropharyngeal airway device permitting oxygen inhalation and sputum aspiration, comprising: an internal airway body 11 extending from a proximal end to a distal end, and a support plate 10 having a width parallel to the lingual surface of the human body, wherein the support plate 10 includes a proximal end 101 and a distal end bending section 102, with the proximal end 101 being located at the front side of the internal airway body 11 and constituting a wall of the tube, and the distal end bending section 102 having a bending angle (α or β) of 0°-180°, wherein the front side of the distal end bending section 102 is provided with at least two uniformly distributed projections 402 (as shown in FIG. 7) or recesses 403 (as shown in FIG. 8) or through holes 401 (as shown in FIG. 6), with the surface of the projection 402 facing to the teeth being smooth and the surface thereof facing to the pharynx having a height of 1-2 mm, or the depth of the recess 403 being 0.5-1 mm or the diameter of the through hole 401 being 1-3 mm.

Compared with the prior art, the efficient care-type oropharyngeal airway device permitting oxygen inhalation and sputum aspiration according to the embodiments of the present disclosure has the following advantages:

The efficient care-type oropharyngeal airway device permitting oxygen inhalation and sputum aspiration provided in the embodiments of the present disclosure can effectively avoid regions in the oropharyngeal cavity sensitive to stimulus, that is to say, it can effectively avoid that regions having a reflected intensity higher than 5 are stimulated, hereby minimizing stimulation area of the sensitive regions mentioned above and reducing stimulation intensity during the anesthesia recovery period. Compared with the traditional oropharyngeal airway device, the efficient care-type oropharyngeal airway device permitting oxygen inhalation and sputum aspiration provided in the embodiments of the present disclosure is safe and efficient in use, and its stimulation area is in a non-sensitive reflection region, hereby reducing the occurrence of nausea and vomiting as well as choking cough, while ensuring an unobstructed glottal upper airway during the anesthesia recovery period. It is especially suitable for patients underwent surgery with general anesthesia, who have acromegaly, snoring disease, glossocoma after extubation and the like, and its purpose is to reduce adverse reactions during the postanesthetic recovery period, conforming to the tool sequential extubation method and the idea of comfort care.

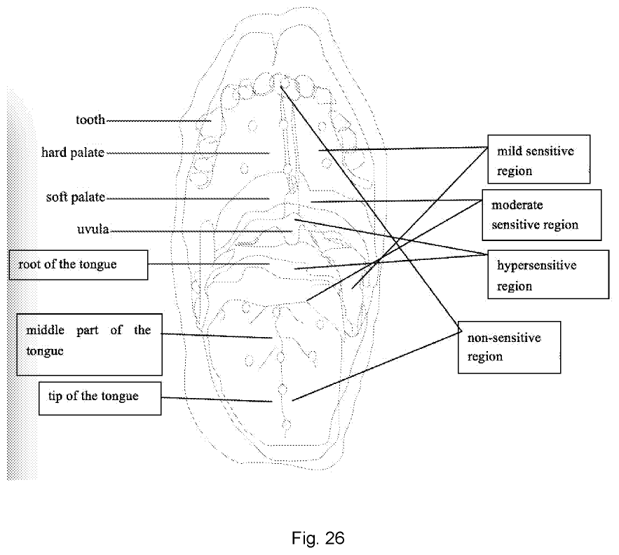

Here, it shall be additionally clarified that the reflected intensity of the oropharyngeal cavity can be evaluated by using the VAS (visual analogue scale). In the oropharyngeal cavity part, teeth have the weakest reflex against stimulation, and the reflected intensity thereof against stimulation is set as 0, while the root of tongue, the posterior pharyngeal wall, and parts close to the laryngeal have the strongest reflex against stimulation, which is set as 10; and large sample data is used in the research method, and a map for sensitivity to stimulus in the oropharyngeal cavity is made based on the above-mentioned data. Sensitive regions in the oropharyngeal cavity are mainly distributed in the pharyngeal cavity, and sensitive regions in the pharyngeal cavity are mainly regions such as at the root of tongue, at the epiglottis, and at the pharyngeal recess, as shown in FIG. 26.

In addition, the tool sequential extubation method is proposed, the content of which is as follows: for the recovery period from general anesthesia during the perioperative period, a set of practical tools is researched, and with the tools, when the patient is recovered from deep anesthesia to shallow anesthesia during the recovery period from general anesthesia, the tools are switched such that the stimulation is changed from heavy to light, so as to eliminate the choking cough reflex, reduce the occurrence of complications, reduce application of anesthetic during the recovery period, and improve the safety of anesthesia during the perioperative period, which is suitable for patients suffering from hypertension, diabetes, adiposis, snoring disease and requiring special surgery. By using this tool sequential extubation method, extubation can be safely executed in sequence during the anesthesia recovery period by means of the tools, without applying analeptics during the anesthesia recovery period, or without additionally applying anesthetics for the purpose of reducing complications caused by stimulations, which might result in a series of risks such as delayed recovery from anesthesia and postanesthetic respiratory depression.

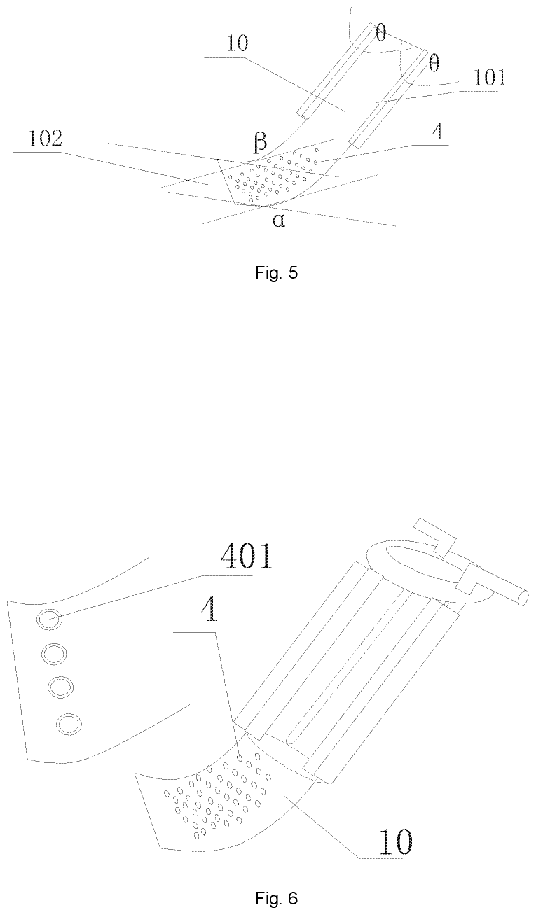

Here, as shown in FIG. 5, the width of the proximal end 101 of the above-mentioned support plate 10 is greater than the width of the distal end bending section 102; and an edgefold is respectively formed at both sides of the proximal end 101, and the included angle θ of the edgefold is in the range of 0°-180°.

In the present embodiment, in order to better integrate an oxygen inhalation function, as shown in FIGS. 1 and 2, the above-mentioned efficient care-type oropharyngeal airway device permitting oxygen inhalation and sputum aspiration further comprises an oxygen inhalation tube 1, which is arranged on a side wall of the internal airway body 11, wherein a side wall of the above-mentioned oxygen inhalation tube 1 is provided with at least two side holes 2 that are spaced from each other, the plurality of side holes 2 are distributed along the proximal end of the internal airway body 11 to the distal end, and a bottom hole at the distal end of the oxygen inhalation tube 1 is closed; and specifically, the perforated angle of the side hole 2 is 0°-180° with respect to any axial lead.

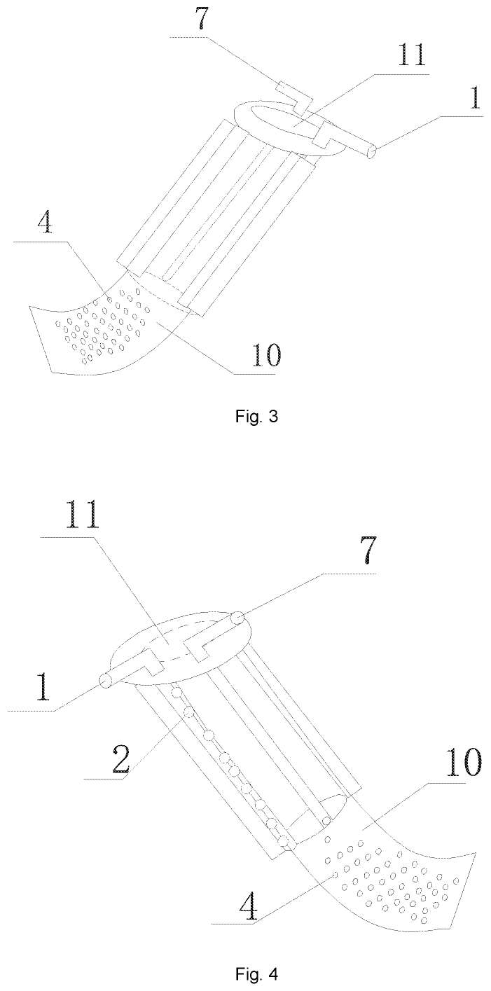

In the present embodiment, in order to better integrate a sputum aspiration function, as shown in FIGS. 3 and 4, the above-mentioned efficient care-type oropharyngeal airway device permitting oxygen inhalation and sputum aspiration further comprises a sputum aspiration tube 7, which is arranged on the side wall of the reverse side of the internal airway body 11 and has a distal opening serving as sputum aspiration hole.

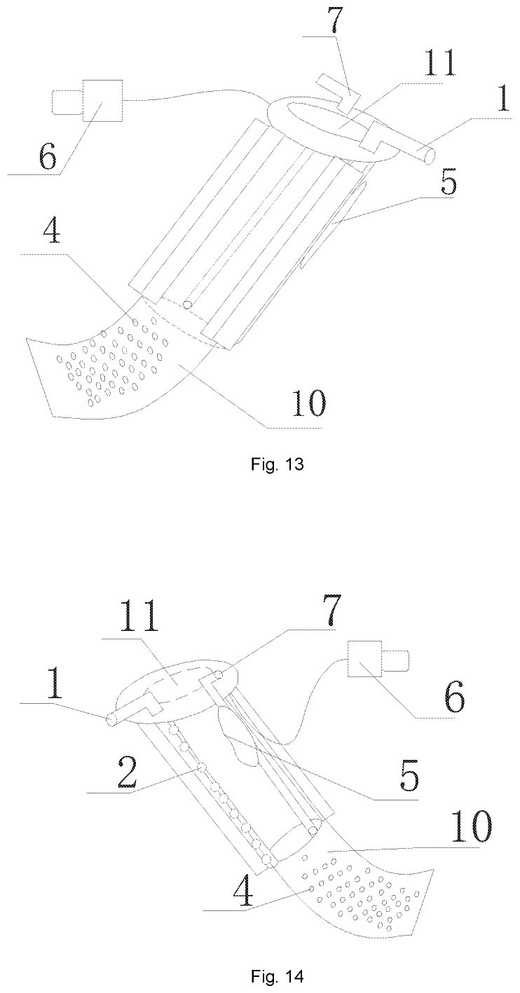

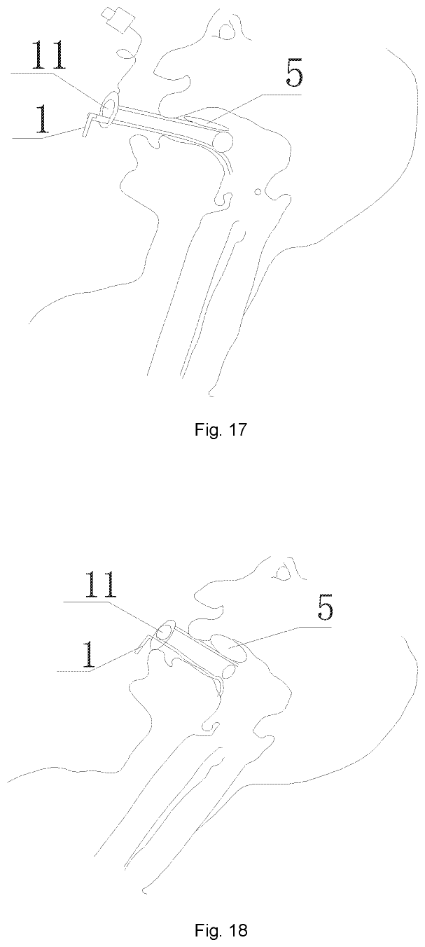

In the present embodiment, as shown in FIGS. 13-16, the above-mentioned efficient care-type oropharyngeal airway device permitting oxygen inhalation and sputum aspiration further comprises an independent air pocket 5, which is positioned on the reverse side of the internal airway body 11 and is in connection with an inflation valve 6. During actual use, as shown in FIGS. 17 and 18, when the independent air pocket 5 is in a non-inflated state (i.e. collapsed state), the efficient care-type oropharyngeal airway device permitting oxygen inhalation and sputum aspiration is inserted into the oropharyngeal cavity; and after inflation, the independent air pocket 5 supports the rear wall of the airway of the efficient care-type oropharyngeal airway device permitting oxygen inhalation and sputum aspiration, and lifts the lingual middle part, hereby keeping the oropharyngeal cavity unobstructed.

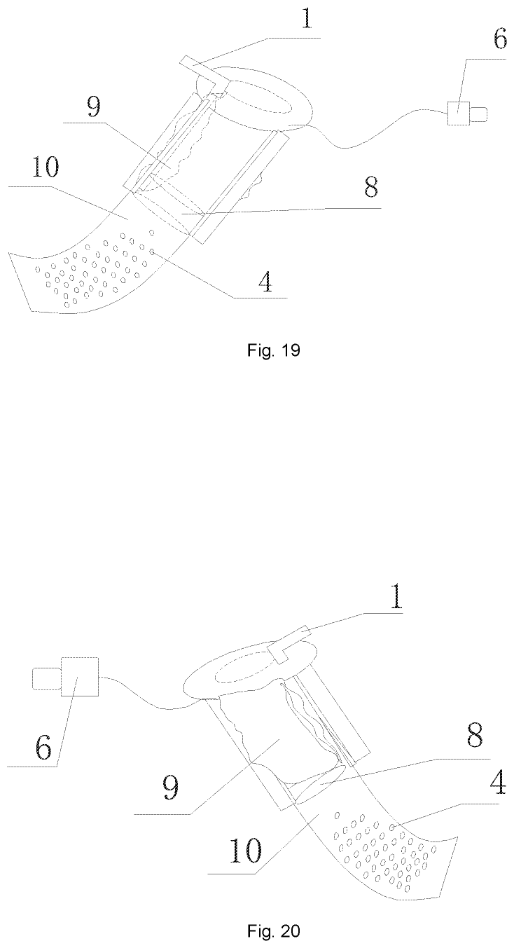

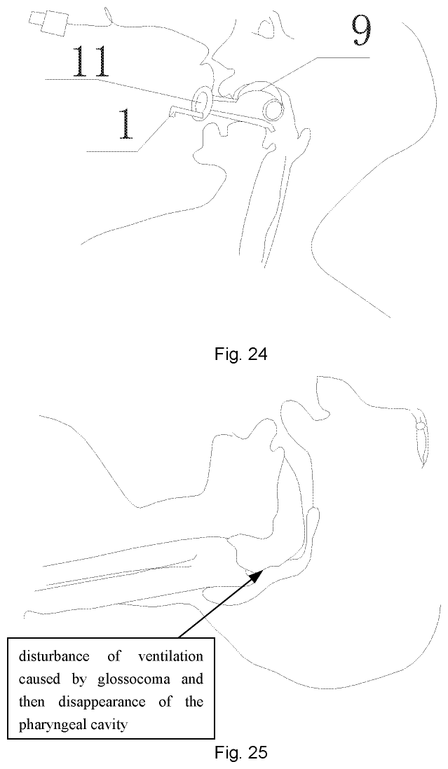

In the present embodiment, as shown in FIGS. 19-22, the above-mentioned efficient care-type oropharyngeal airway device permitting oxygen inhalation and sputum aspiration further comprises a double-layered air pocket 9, which is positioned on the reverse side of the internal airway body 11 and constitutes the rear wall of the internal airway body 11. Specifically, the double-layered air pocket 9 is in communication with the inflation valve 6, and the double-layered air pocket 9 is in sealed connection with the hard tube wall and a tubal ring 8. When the double-layered air pocket 9 is in a non-inflated state (i.e. collapsed state), the efficient care-type oropharyngeal airway device permitting oxygen inhalation and sputum aspiration is inserted into the oropharyngeal cavity, as shown in FIG. 23; and after inflation, the double-layered air pocket 9 supports the rear wall of the airway of the efficient care-type oropharyngeal airway device permitting oxygen inhalation and sputum aspiration, and lifts the lingual middle part, hereby keeping the oropharyngeal cavity unobstructed, as shown in FIG. 24.

In the present embodiment, in the efficient care-type oropharyngeal airway device permitting oxygen inhalation and sputum aspiration provided in the embodiments of the present disclosure, the above-mentioned internal airway body 11 may have a circular cross-section or an elliptical cross-section or a special-shaped cross-section. Of course, no limitation is made here regarding the specific cross-sectional shape, as long as it is reasonable.

The efficient care-type oropharyngeal airway device permitting oxygen inhalation and sputum aspiration provided in the embodiments of the present disclosure can be manufactured by adopting injection molding or rotational molding or other types of molding processes.

An embodiment of the present disclosure further provides an efficient care-type oropharyngeal airway device permitting oxygen inhalation and sputum aspiration, as shown in FIGS. 1-24, comprising: an internal airway body 11 extending from a proximal end to a distal end, and a support plate 10 having a width and parallel to the lingual surface of the human body, wherein the support plate 10 has a plate-like proximal end 101, and the both sides of the plate are infolded with an infolding radian of 0°-180°, while the distal end of the support plate 10 is a curved arc structure with a bending radian of 0°-180°; the curved arc structure of the distal end of the support plate 10 is provided with an tongue adhering protuberance 4, which includes through holes 401 or projections 402 or recesses 403; the internal airway body 11 has a length having a ratio of 1.5/3-2.5/3 with respect to the overall length of the efficient care-type oropharyngeal airway device permitting oxygen inhalation and sputum aspiration; the internal airway body 11 is in a hard circular or elliptical structure, or the internal airway body 11 is in a soft special-shaped structure; the internal airway body 11 is in communication with an inflation valve 6, and the reverse side of the hard circular or elliptical structure is provided with an inflation pocket constituting a tubal cavity part and being in communication with the inflation valve 6; alternatively, the soft special-shaped structure is adhered to the hard part and is in communication with a tubal ring 8 and the inflation valve 6, and constitutes a tube wall of the efficient care-type oropharyngeal airway device permitting oxygen inhalation and sputum aspiration when being inflated; a side tube wall of the internal airway body 11 is provided with an oxygen inhalation tube 1, which is provided with at least two side holes 2, wherein every two airflows respectively between two adjacent side holes 2 form an included angle therebetween and converge in the internal of the internal airway body 11 to form a turbulence; the bottom of the oxygen inhalation tube 1 is closed; and the back tube wall of the internal airway body 11 is provided with a sputum aspiration tube 7.

The metabolism of anesthetic drugs during the anesthesia recovery period is a slow process. Anesthetics and metabolites thereof remaining in the body will continue to play an anesthetic role, which manifests under strong stimulation intolerance of stimulations, hereby causing complications during the anesthesia recovery period. However, a premature withdrawal of the tracheal catheter causes that the patient continues to enter lethargic or anesthesia state because of the residual effect of anesthetics, and common complications include airway obstruction due to muscular flaccidity in the pharyngeal cavity and glossocoma. It is a reasonable choice to implant an oropharyngeal airway having a smaller stimulation intensity after withdrawing a more irritating tracheal catheter during the anesthesia recovery period. As for traditional oropharyngeal airway devices, sensitive parts in the oropharyngeal cavity are not avoided in design, thus, it would easily render new adverse reflex. In the efficient care-type oropharyngeal airway device permitting oxygen inhalation and sputum aspiration provided in the present disclosure, adverse reflex in the oral cavity can be avoided or reduced, meanwhile, the integration of sputum aspiration function and oxygen inhalation function in the efficient care-type oropharyngeal airway device permitting oxygen inhalation and sputum aspiration facilitates sputum aspiration as well as oxygen inhalation operations during the clinical anesthetizing process. The tool sequential extubation method is applied to safely realize extubation in sequence by combining advantages of several novel tools, with reduced stimulation, and to reduce damages caused by operations of supplementary anesthetic drugs in traditional anesthetizing process for the purpose of inhibiting tool stimulation; meanwhile, the efficiency of tools can be improved, and problems that cannot be solved by traditional tools are solved, for example, safe ventilation during the postanesthetic recovery period of patients underwent hypertension, diabetes, adiposis, snoring disease and special surgery.

The above mentioned is merely preferred embodiments of the present disclosure, and is not intended to limit the present disclosure. Any modifications, equivalent substitutions and improvements made within the spirit and principle of the present disclosure shall all be covered in the scope of protection of the present disclosure.

### INDUSTRIAL APPLICABILITY

The efficient care-type oropharyngeal airway device permitting oxygen inhalation and sputum aspiration provided in the embodiments of the present disclosure can effectively avoid regions in the oropharyngeal cavity sensitive to stimulus, that is to say, it can effectively avoid that regions having a reflected intensity higher than 5 are stimulated, hereby minimizing stimulation area of the sensitive regions mentioned above and reducing stimulation intensity during the anesthesia recovery period. Compared with the traditional oropharyngeal airway device, the efficient care-type oropharyngeal airway device permitting oxygen inhalation and sputum aspiration provided in the embodiments of the present disclosure is safe and efficient in use, and its stimulation area is in a non-sensitive reflection region, hereby reducing the occurrence of nausea and vomiting as well as choking cough, while ensuring an unobstructed glottal upper airway during the anesthesia recovery period. It is especially suitable for patients underwent surgery with general anesthesia, who have acromegaly, snoring disease, glossocoma after extubation and the like, and its purpose is to reduce adverse reactions during the postanesthetic recovery period, conforming to the tool sequential extubation method and the idea of comfort care. The tool sequential extubation method is as follows: for the recovery period from general anesthesia during the perioperative period, a set of practical tools is researched, and with the tools, when the patient is recovered from deep anesthesia to shallow anesthesia during the recovery period from general anesthesia, the tools are switched such that the stimulation is changed from heavy to light, so as to eliminate the choking cough reflex, reduce the occurrence of complications, reduce reapplication of anesthetics and delay of recovery during the recovery period, and improve the safety of anesthesia during the perioperative period, and be suitable for patients suffering from hypertension, diabetes, adiposis, snoring disease and requiring special surgery.

1. An efficient care-type oropharyngeal airway device permitting oxygen inhalation and sputum aspiration, comprising: an internal airway body extending from a proximal end to a distal end, and a support plate having a width and parallel to a lingual surface of a human body,
wherein the support plate comprises a proximal end and a distal end bending section, with the proximal end being located at a front side of the internal airway body and constituting a wall of a tube, and the distal end bending section having a bending angle of 0°-180°,
wherein a front side of the distal end bending section is provided with at least two uniformly distributed projections or recesses or through holes, with a surface of the projections facing to teeth being smooth and a surface thereof facing to a pharynx having a height of 1-2 mm, or a depth of the recesses being 0.5-1 mm or a diameter of the through holes being 1-3 mm.

2. The efficient care-type oropharyngeal airway device permitting oxygen inhalation and sputum aspiration according to claim 1, wherein a width of the proximal end of the support plate is greater than a width of the distal end bending section; and an edgefold is respectively formed at both sides of the proximal end, and an included angle is in a range of 0°-180°.

3. The efficient care-type oropharyngeal airway device permitting oxygen inhalation and sputum aspiration according to claim 1, further comprising an oxygen inhalation tube, which is arranged on a side wall of the internal airway body.

4. The efficient care-type oropharyngeal airway device permitting oxygen inhalation and sputum aspiration according to claim 3, wherein a side wall of the oxygen inhalation tube is provided with at least two side holes that are spaced from each other, a plurality of the side holes are distributed along the proximal end of the internal airway body to the distal end, and a bottom hole at a distal end of the oxygen inhalation tube is closed; and
a perforated angle of the side holes is 0°-180° with respect to any axial lead.

5. The efficient care-type oropharyngeal airway device permitting oxygen inhalation and sputum aspiration according to claim 1, further comprising a sputum aspiration tube, which is arranged on a side wall of a reverse side of the internal airway body and has a distal opening serving as a sputum aspiration hole.

6. The efficient care-type oropharyngeal airway device permitting oxygen inhalation and sputum aspiration according to claim 1, further comprising an independent air pocket, which is located on the reverse side of the internal airway body and is in connection with an inflation valve.

7. The efficient care-type oropharyngeal airway device permitting oxygen inhalation and sputum aspiration according to claim 1, further comprising a double-layered air pocket, which is located on the reverse side of the internal airway body and constitutes a rear wall of the internal airway body.

8. The efficient care-type oropharyngeal airway device permitting oxygen inhalation and sputum aspiration according to claim 1, wherein the internal airway body has a circular cross-section, an elliptical cross-section or a special-shaped cross-section.

9. An efficient care-type oropharyngeal airway device permitting oxygen inhalation and sputum aspiration, comprising: an internal airway body extending from a proximal end to a distal end, and a support plat having a width and parallel to a lingual surface of a human body,
wherein the support plate has a plate-like proximal end, and both sides of the plate are infolded with an infolding radian of 0°-180°, and a distal end of the support plate is a curved arc structure with a bending radian of 0°-180°;
the curved arc structure of the distal end of the support plate is provided with a tongue adhering protuberance, which comprises through holes, projection or recesses;
the internal airway body has a length having a ratio of 1.5/3-2.5/3 with respect to an overall length of the efficient care-type oropharyngeal airway device permitting oxygen inhalation and sputum aspiration; the internal airway body is in a hard circular or elliptical structure, or the internal airway body is in a soft special-shaped structure;
the internal airway body is in communication with an inflation valve, and a reverse side of the hard circular or elliptical structure is provided with an inflation pocket, which constitutes a tubal cavity part and is in communication with the inflation valve; alternatively, the soft special-shaped structure is adhered to a hard part and is in communication with a tubal ring and the inflation valve, and when being inflated, constitutes a tube wall of the efficient care-type oropharyngeal airway device permitting oxygen inhalation and sputum aspiration;
a side tube wall of the internal airway body is provided with an oxygen inhalation tube, which is provided with at least two side holes, wherein every two airflows respectively between two adjacent side hole form an included angle therebetween and converge in an internal of the internal airway body to form a turbulence; a bottom of the oxygen inhalation tube is closed; and
the back tube wall of the internal airway body is provided with a sputum aspiration tube.
